(19) United States
(12) Patent Application Publication
(10) Pub. No.: US 2006/0153893 A1
(43) Pub. Date: Jul. 13, 2006

(54) Therapeutic composition for bone infectious disease

(75) Inventors: Hiroaki Matsuno, TOYAMA (JP); Masamichi Hashimoto, Tokyo (JP); Yoshiaki Miyata, Tokyo (JP); Teruzuo Miyoshi, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: DENKI KAGAKU KOGYO KABUSHIKI KAISHA, TOKYO (JP)

(21) Appl. No.: 10/509,323

(22) PCT Filed: Apr. 8, 2003
(86) PCT No.: PCT/JP03/04433

§ 371 Date: May 31, 2005

(30) Foreign Application Priority Data
Apr. 8, 2002 (JP) 2002-105133

(51) Int. Cl. A61K 38/14; A61K 31/728; A61K 31/7034; A61F 2/00

(52) U.S. Cl. 424/426; 514/8; 514/35; 514/54

(57) ABSTRACT

A biodegradable composition containing an antibiotic or a physiologically active substance for use in surgical treatment of infection. A highly safe and biocompatible composition showing appropriately sustained release of an antibiotic or physiologically active substance which produces excellent antibiotic and bone regenerating effects. A composition having excellent effects in treatment of bone infection occurring after operations for total arthroplasty and/or bone fracture. (1) A medical composition for treatment of bone infection comprising an antibiotic and a polysaccharide.

### TECHNICAL FIELD

The present invention relates to a composition for treatment of bone infection, comprising an antibiotic and a polysaccharide, and preferably further comprising a physiologically active substance, namely a composition for treatment of bone infection. In particular, it relates to a composition for treatment of bone infection such as orthopedic bone infection or nonsurgical acute/chronic osteomyelitis, which comprises an antibiotic such as gentamicin, a biodegradable polysaccharide such as hyaluronic acid and/or a hyaluronic acid gel, and preferably further comprising a physiologically active substance.

### BACKGROUND ART

Bone infection occurring after total arthroplasty and/or fracture surgery and nonsurgical acute or chronic bone infection are a serious problem which imposes heavy time and financial burdens on the patients and the medical institutions.

Infections after total arthroplasty and/or fracture surgery develop into pyogenic bone infection called osteomyelitis. Osteomyelitis causes systemic bacteremic symptoms such as fever, chill, nausea and dehydration and local symptoms such as pain, tenderness, heat and local sequestration, which can lead to pseudarthrosis and fistulization.

For prevention of bone infection, not only radical treatment of infected lesions, preoperative/postoperative antibiotic medication and sterilization of surgical instruments/gowns are carried out. However, even with aseptic surgery in bioclean rooms, it is difficult to entirely prevent bone infections.

Despite such surgical techniques, complete prevention of infection is difficult because most total arthroplasty patients are elderly people with reduced immunity or patients with articular rheumatism, and the overall surgical infection rate amounts to about 1%.

There is no established treatment yet, and ordinary treatment comprises removal of artificial joints, curettage/irrigation of the infected lesions and antibiotic washing. Bone infections are usually difficult to cure, and when complete healing is difficult even with removal of artificial joints, some patients have to undergo great suffering such as amputation. The need for prolonged hospitalization and high medical expenses in treatment of bone infections is a social issue which heavily burdens not only patients but also medical institutions and medical finances.

Nonsurgical traumatic acute/chronic osteomyelitis is also intractable and is known to be difficult to completely cure even with repetitive irrigation and curettage of infected lesions in some cases.

Nonsurgical hematogenous chronic osteomyelitis is also known to have a poor prognosis with a possibility of inducing osteonecrosis which leads to pseudarthrosis and capitular necrosis. Patients are still getting socially problematic treatment such as amputation and arthrodesis.

No effective treatment is available for any type of bone infections at present irrespective of the causation, and only palliative treatment is given with unsatisfactory results.

Such treatment involves antibiotic medication. Systemic administration requires high doses of antibiotics to secure effective local antibiotic concentrations with the high possibility of causing problems such as serious side effects and emergence of drug-resistant strains of bacteria.

For local treatment of bone infections after total arthroplasty, poly(methyl methacrylate) (bone cement) blended with antibiotics has been implanted in infected lesions.

In this treatment, an admixture of bone cement and antibiotics molded into a chain of beads is laid in infected lesions in joints and the bone marrow for a long time, and it is especially suitable as local antibiotic chemotherapy.

However, because bone cement is a foreign matter which cannot be absorbed in the body, it has to be removed again and presumably cannot sustain the release of antibiotics sufficiently. Treatment using cement beads comprises, for example, implanting a chain of beads with either end sticking out through the skin at a suture and pulling out the entire chain by the end when the treatment finishes about two weeks later.

During about two weeks between the insertion and removal of beads, patients require fixation of the affected parts, rest in bed and hospitalization. Therefore, there are problems of pain and heavy financial burdens.

To overcome the drawback of necessary removal of bone cement, attempts to design dosage forms using biodegradable supports which can enhance the effect of antibiotics have been made.

Bioabsorbable materials having readily controllable bioabsorbability and high biocompatibility are preferred, and for example, biogenic proteins such as fibrin glue, collagen and gelatin may be mentioned. Further, polylactic acid obtained by polymerization of the organic acid, lactic acid, may be mentioned.

For example, fibrin glue is a biological adhesive utilizing solidification of a fibrinogen solution upon addition of thrombin based on the mechanism of blood coagulation. It is known that fibrin glue is used to repair bone defects in regions of transplantation as a sealant and is as a surgical adhesive. A fibrin/antibiotic gel for treatment of bone infections and its preparation are disclosed as an approach to treatment using a mixture of fibrin glue with gentamicin as a sealant in infected lesions (JP-B-56-501129 and JP-A-8-502161).

However, because fibrin glue utilizes the mechanism of blood coagulation, it is basically difficult to control the biodegradation time arbitrarily, though it is possible to control the gelation time. Therefore, it is difficult to retain drugs in the target region at an effective concentration for an appropriate period of time.

Besides, though solidification of fibrin glue gives a hard gel which efficiently releases the drug from the surface, there is a problem that the gel is unlikely to show sustained release of drugs from inside. Moreover, because fibrin glue is a blood product prepared from human blood, the risk of serving as a source of transmission of hepatitis C, AIDS, and other unknown viruses cannot be eliminated.

Collagen and gelatin as major proteins constituting the body are especially suitable as a bioabsorbable material and has been used as a substrate for bone and cartilage regeneration in the field of regenerative medicine in recent years. An attempt to treat bone infections by filling these crosslinked gelatin gels blended with antibiotics into infected lesions is disclosed as a therapy for osteomyelitis (U.S. Pat. No. 4,587,268).

However, because of the ingredients of animal origin, the risk of serving as a source of transmission of bovine spongiform encephalopathy and other unknown viruses cannot be eliminated. These heterologous proteins including atelocollagen cannot be escaped from the problem of antigenicity even after reduction in antigenicity.

Polylactic acid obtained by polymerization of lactic acid as an organic acid has been used for development of bioabsorbable materials such as bioabsorbable bone crews for implantation in recent years. Treatment of infected lesions with microcapsules of polylactic acid loaded with antibiotics is disclosed as a therapy for osteomyelitis (U.S. Pat. No. 6,309,699).

However, because polylactic acid is physically rigid and brittle in essence, its application is limited. Further, the pH sift to the acidic side due to lactic acid produced as the biodegradation product can be hazardous to the healing of the lesions.

There have been reports on various compositions containing antibiotics and polysaccharides such as eye drops containing hyaluronic acid and antibiotics such as streptomycin and penicillin for ophthalmic use (JP-A-60-84225), a bone replacement containing hyaluronic acid or its derivative and antibiotics used for defective bones in the field of orthopedics (WO93/20858), hyaluronic acid loaded with antibiotics for prevention and treatment of celiac infection (JP-A-09-208476), a cornea stock medium containing hyaluronic acid and antibiotics (JP-A-2000-508637) and a therapeutic material for arthritis containing hyaluronic acid or its derivative and antibiotics (JP-A-2000-512650).

Heretofore, no compositions using polysaccharides, especially biodegradable polysaccharides, with controlled release of antibiotics for treatment of bone infections, especially infections of artificial joints have been developed yet. We extensively studied the possibility of application of polysaccharides, especially biodegradable polysaccharides to treatment of infections caused by orthopedic surgery and have found that biodegradable polysaccharides such as hyaluronic acid and carboxymethylcellulose are extremely useful. The present invention has been accomplished based on this discovery.

### DISCLOSURE OF THE INVENTION

The present invention provides (1) a composition for treating bone infection, which comprises an antibiotic and a polysaccharide, (2) the composition according to (1), wherein the bone infection is traumatic bone infection, (3) the composition according to (1), wherein the bone infection is hematogenous bone infection, (4) the composition according to (1), wherein the bone infection occurs after total arthroplasty and/or fracture surgery, (5) the composition according to any one of (1) to (4), wherein the polysaccharide is a biodegradable polysaccharide and/or a polysaccharide gel, (6) the composition according to (5), wherein the polysaccharide is an acidic polysaccharide, (7) the composition according to (6), wherein the acidic polysaccharide is hyaluronic acid and/or a hyaluronic acid gel, (8) the composition according to (6), wherein the acidic polysaccharide is carboxymethylcellulose and/or a carboxymethylcellulose gel, (9) the composition according to (7), wherein the hyaluronic acid gel is crosslinked hyaluronic acid made of hyaluronic acid having a weight average primary molecular weight greater than 800,000, (10) the composition according to (9), wherein the crosslinks in the crosslinked hyaluronic acid are hydrolysable, (11) the composition according to (9) or (10), wherein the crosslinks in the crosslinked hyaluronic acid have ester linkages in the structure, (12) the composition according to (11), wherein the crosslinks in the crosslinked hyaluronic acid have self-ester linkages in the structure, (13) the composition according to any one of (1) to (12), wherein the antibiotic is one member selected from the group consisting of gentamicin, vancomycin and minomycin, (14) the composition according to (13), which is in the form of one member selected from the group consisting of a sheet, a film, a rod, a sponge, a mass, a fiber, a paste, a gel suspension and a tube.

### BRIEF DESCRIPTION OF THE DRAWINGS

FIG. 1: A radiograph of a mouse femur 4 weeks after MSSA injection and implantation of a K-wire wrapped with a HA gel sheet containing 10 mg GM obtained in Example 3.

FIG. 2: A radiograph of a mouse femur 4 weeks after MSSA injection and implantation of a bare K-wire.

FIG. 3: The cell counts per 1 mg of tissue samples from around mouse femurs 1 week after the operation.

FIG. 4: The change with time in the cell counts per 1 mg of tissue samples from around mouse femurs.

FIG. 5: A top radiograph of a rabbit femur 8 weeks after implantation of a stem wrapped with a HA gel sheet containing 10 mg GM obtained in Example 3 without MSSA injection.

FIG. 6: A lateral radiograph of a rabbit femur 8 weeks after implantation of a stem wrapped with a HA gel sheet containing 10 mg GM obtained in Example 3 without MSSA injection.

FIG. 7: A top radiograph of a rabbit femur 8 weeks after MSSA injection and implantation of a stem wrapped with a GM-free HA gel sheet obtained in Comparative Example 1.

FIG. 8: A lateral radiograph of a rabbit femur 8 weeks after MSSA injection and implantation of a stem wrapped with a GM-free HA gel sheet obtained in Comparative Example 1.

FIG. 9: A top radiograph of a rabbit femur 8 weeks after MSSA injection and implantation of a stem wrapped with a HA gel sheet containing 10 mg GM obtained in Example 3.

FIG. 10: A lateral radiograph of a rabbit femur 8 weeks after MSSA injection and implantation of a stem wrapped with a HA gel sheet containing 10 mg GM obtained in Example 3.

FIG. 11: A pathologic tissue specimen of a rabbit femur 8 weeks after implantation of a bare stem without MSSA injection.

FIG. 12: A pathologic specimen of a rabbit femur 8 weeks after implantation of a stem wrapped with a HA gel containing 10 mg GM obtained in Example 3 without MSSA injection.

FIG. 13: A pathologic tissue specimen of a rabbit femur 8 weeks after MSSA injection and implantation of a stem wrapped with a GM-free HA gel sheet obtained in Comparative Example 1.

FIG. 14: A pathologic tissue specimen of a rabbit femur 8 weeks after MSSA injection and implantation of a stem wrapped with a HA gel sheet containing 10 mg GM obtained in Example 3.

FIG. 15: Retention of vancomycin in the bone marrow after implantation of a vancomycin solution (3 mg/500 μl) and a freeze-dried HA gel sponge impregnated with vancomycin (300 mg vancomycin/4 mm×Ø4 mm) into rabbit arthroplasty models.

FIG. 16: Retention of VM, MC and GM in the bone marrow after implantation of VM and MC-impregrated HA gel sponges and GM-containing collagen sponge into rabbit arthroplasty models.

FIG. 17: Retention of VM after implantation of HA gel film-wrapped metal rods into rabbit arthroplasty models.

### EXPLANATION OF THE SYMBOLS

(A) Fracture healing

(B) Pseudathrosis

(C) K-wire

(D) Stem

(E) Osteomyelitis

(F) Bone extension

(G) Abscess formation

### MODE FOR CARRYING OUT THE INVENTION

Now, the present invention will be described in detail.

In the present invention, polysaccharides isolated from animal/plant tissues or obtained by fermentation may be used irrespective of their origin. Those having biodegradability, substantially no antigenicity and high biocompatibility are preferred. In the present invention, the concept of polysaccharides includes their salts with alkali metals such as sodium, potassium or lithium.

Examples of the polysaccharide used in the present invention include glycosaminoglycans (such as hyaluronic acid, heparin, heparin sulfate and dermatan sulfate), chondroitin sulfates (such as chondroitin-6-sulfate), keratin sulfate, heparin, heparin sulfate, alginic acid or its biologically acceptable salts, cellulose, chitin, chitosan, dextran, starch, amylose, carrageenan and the like. Also, synthetic polysaccharide derivatives such as carboxymethylcellulose, carboxymethylamylose, various alkylcellulose, hydroxyethylcellulose, carboxycellulose or oxidized regenerated cellulose may be mentioned.

In the present invention, as a biodegradable polysaccharide, an acidic polysaccharide is especially preferable in view of reactivity during gelation of polysaccharides. While neutral polysaccharides are rich in hydroxyl group, acidic polysaccharides are rich in uronic acids, sulfate groups and carboxyl groups, which are more reactive than hydroxyl groups, and therefore suitable for chemical reactions involved in gelation of polysaccharides.

In the present invention, a biodegradable acidic polysaccharide such as hyaluronic acid or carboxymethylcellulose may be used. Hyaluronic acid is a major and common component of the extracellular matrix which acts as a cell lubricant, adhesive and scaffold in animals including human and a linear polymer consisting of alternately bonded β-D-N-acetylglucoamine and β-D-glucuronic acid present in synovial fluid, the vitreous humor of the eye and rooster combs in large amounts. Being a component of the human body, hyaluronic acid is substantially free of antigenicity and ideally biocompatible. Therefore, it is used in therapeutic agents for knee osteoarthritis, in adjuvants for eye surgery and in adhesion preventives.

The polysaccharide gel to be used in the present invention is preferably a highly biocompatible acidic polysaccharide gel such as a hyaluronic acid gel, though it is not particularly limited.

A gel is defined as “a polymer having a three-dimensional network structure insoluble in any solvent or its swollen product” by Encyclopedia of Polymer (Kobunshi Jiten) New Edition (published by Asakura Shoten, 1988). It is also defined as “a jellied product of a sol (a colloidal solution)” by Encyclopedia of Science and Chemistry (Rikagaku Jiten) Forth Edition (published by Iwanami Shoten, 1987).

Representatives of them are crosslinked hyaluronic acid gels obtained by crosslinking the acidic polysaccharide, hyaluronic acid, with a bifunctional crosslinker such as divinyl sulfone, a bisepoxide or formaldehyde (U.S. Pat. No. 4,582,865, JP-B-6-37575, JP-A-7-97401 and JP-A-60-130601).

The present inventors proposed a method of producing a hyaluronic acid gel by crosslinking hyaluronic acid without impairing the ideal characteristics intrinsic to hyaluronic acid as a biomaterial (WO99/10385) and found the following facts.

Namely, a hardly water-soluble hyaluronic acid gel and a hardly water-soluble carboxymethylcellulose gel (PCT/JP/05564) obtained by gelation using no crosslinkers are particularly preferable in view of biocompatibility and safety because no crosslinker is used for the gelation.

Further, studies on the molecular structures of the crosslinked hyaluronic acids and the production conditions revealed that crosslinked hyaluronic acid obtained from hyaluronic acid having a weight average primary molecular weight higher than 800,000 without impairing the excellent properties of hyaluronic acid (Japanese Patent Application No. 2002-314090) is preferable in view of controlled retention of antibiotics and physiologically active substances.

The hydrogel of crosslinked hyaluronic acid has chains of hyaluronic acid macromolecules on the surface and various properties of hyaluronic acid macromolecules such as a high holding capacity for various cytokines, especially positively charged cytokines, attributable to its strong ionic interaction with them as a negatively charged polymer electrolyte.

A composition obtained by loading the crosslinked hyaluronic acid thus obtained with antibiotics or physiologically active substances highly promotes regeneration of bone tissues by virtue of the high affinity of the crosslinked hyaluronic acid hydrogel containing hyaluronic acid macromolecules for bone defects.

The composition of the present invention for treating bone infection comprises a polysaccharide and an antibiotic such as those as mentioned above so that the polysaccharide/antibiotic ratio is preferably from 1:9 to 9:1.

The rate at which the composition of the present invention for treating bone infection releases antibiotics and physiologically active substances and the bioabsorption rate of the composition of the present invention can be varied by changing the conditions for its production such as the molecular weight and concentration of the polysaccharide and the polysaccharide gel, the type and amount of the crosslinking agent and the reaction time.

For example, the hardly water-soluble hyaluronic acid (WO99/10385) can be obtained so that it releases antibiotics and physiologically active substances at various rates and is bioabsorbed at various rates, depending on the conditions such as the molecular weight and concentration of the hyaluronic acid.

The composition of the present invention holds antibiotics and physiologically active substances in it by inonic bonding, hydrogen bonding or covalent bonding without impairing their activities.

When the interaction between the polysaccharide and/or polysaccharide gel and the antibiotic or physiologically active substance such as ionic bonding or hydrogen bonding is relatively strong, the antibiotic or physiologically active substance is released by biodegradation of the polysaccharide and/or polysaccharide gel.

On the other hand, when the interaction between the polysaccharide and/or polysaccharide gel and the antibiotic or physiologically active substance such as ionic bonding or hydrogen bonding is relatively weak, the antibiotic or physiologically active substance is held in the swollen polysaccharide polysaccharide having a high water content. Therefore, the antibiotic or physiologically active substance is released by diffusion of the antibiotic or physiologically active substance depending on the concentration gradient and by biodegradation of the polysaccharide and or polysaccharide gel.

The retention time of the antibiotic or physiologically active substance in the composition of the present invention can be controlled by changing the biodegradability of the polysaccharide gel or by adjusting the noncovalent interaction with the antibiotic or physiologically active substance by changing the type or concentration of the polysaccharide.

As the antibiotic to be used in the present invention, gentamicin, which shows a broad antibiotic spectrum against both gram-negative and gram-positive bacteria, may be mentioned. Gentamicin is an aminoglycoside antibiotic commonly used in surgical operations as a topical medicament by parenteral administration.

As examples of β-lactam antibiotics, penicillin antibiotics such as ampicillin, amoxicillin, penicillin G, carbenicillin, tacarcillin and methicillin, cephalosporin antibiotics such as celaclor, cefarodxil, cefamandole, cefazolin and cefaperazone and other β-lactam antibiotics such as aztreonam and imipenem may be mentioned.

As macrolide antibiotics, erythromycin or the like may be mentioned. As examples of aminoglycoside antibiotics, streptomycin, neomycin, lincomycin, kanamycin, vancomycin, sisomycin and the like may be mentioned.

As examples of polypeptide antibiotics, bacitracin and novobiocin may be mentioned.

The composition of the present invention may be prepared in a dry state, depending on the intended use, by air-, vacuum- or freeze-drying a polysaccharide and/or polysaccharide gel impregnated with an antibiotic solution or with a polysaccharide solution containing an antibiotic or physiologically active substance.

The composition of the present invention may be available in a wet state impregnated with an antibiotic or physiologically active substance solution or a polysaccharide solution containing an antibiotic or physiologically active substance.

The composition of the present invention may be in the form of one member selected from the group consisting of a sheet, a film, a rod, a sponge, a mass, a fiber, a paste, a gel suspension and a tube.

As examples of the physiologically active substance in the composition of the present invention, the following pharmacologically or physiologically active substances may be mentioned. For example, it may be a mixture or combination with physiologically active substances which stimulate osteogenic healing such as BMP and TGF without any restrictions.

As physiologically active substances, factors which stimulate growth of osteocytes such as BMP, FGF, VEGF, HGF, TGF, CSF, EPO, IL and IF may be mentioned. These physiologically active substances may be prepared through recombinant technology or isolated from protein mixtures. BMP includes rhBMP-2, rhBMP-3, rhBMP-4, rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12 and rhGDF-14, and they are collectively called the BMP family. Further, they may be used in the form of homodimers, heterodimers, modified products, partial deletion products or mixtures of two or more of them, such as the heterodimer of BMP and another member of the TGF-β superfamily such as activin, inhibin or TGF-β1.

The composition of the present invention is used for treatment of bone infections in various fields and suitable especially for bone infections after total arthroplasty or fracture surgery.

For example, in total hip arthroplasty, it is preferably applied in the form of a sheet, a film or a sponge to the socket component, or in the form of a sheet or a film to the femur component. For filling into the medullary cavity, it is preferably used in the form of a sheet, a film, a sponge, a mass, a fiber, a paste or a gel suspension.

In the present invention, a crosslinked hyaluronic acid obtained from hyaluronic acid having a weight average primary molecular weight higher than 800,000 means that cleavage of the crosslinks in the crosslinked hyaluronic acid gives linear hyaluronic acid molecules having an average molecular weight higher than 800,000. The weight average molecular weight and branching degree of the hyaluronic acid obtained after cleavage of the crosslinks are measured readily by GPC-MALLS.

Since synthesis of crosslinked hyaluronic acid has aimed at improved retention in the body so far, the molecular weight of the hyaluronic acid molecules constituting the crosslinked hyaluronic acid has never been considered or actually measured in any studies.

In the present invention, it is meant by “the crosslinks in the crosslinked hyaluronic acid are hydrolysable” that under physiological conditions, for example, at 37° C. at pH 7.4 in physiological saline, the cleavage of crosslinks predominates over the cleavage of the main chain.

The crosslinks which are hydrolysable than the main chain of hyaluronic acid have carbamate linkages, hydrazone linkages, hydrazide linkages or phosphate ester linkages, typically ester linkages, in the structure.

Examples of crosslinked hyaluronic acid having ester linkages in the crosslinks include hyaluronic acid having carboxyl groups esterified with a polyhydric alcohol, hyaluronic acid having hydroxyl groups esterified with a polycarboxylic acid, and hyaluronic acid having carboxyl group esterified with a polyepoxy compound.

The crosslinked hyaluronic acid having self-ester linkages in the crosslinks means crosslinked hyaluronic acid in which carboxyl groups and hydroxyl groups in hyaluronic acid molecules have formed ester linkages.

For preparation of crosslinked hyaluronic acid having self-ester linkages in the corsslinks, hyaluronic acid having self-ester linkages in the crosslinks in which part or all of the carboxy groups in one polysaccharide chain are esterified with alcohol groups in the same or different polysaccharide chain is disclosed in EP0341745B1, and hyaluronic acid having self-ester linkages in the crosslinks obtained by acidification of a hyaluronic acid aqueous solution followed by freezing and thawing at least once is disclosed in WO99/10385.

Crosslinked hyaluronic acid having self-ester linkages in the crosslinks can be safer than crosslinked hyaluronic acid obtained through a different crosslinking reaction, because its hydrolysis product is the naturally occurring hyaluronic acid, which is metabolized in the physiological metabolic pathway.

Now, the present invention will be described in further detail with reference to Examples. However, the present invention is by no means restricted to these specific Examples.

### EXAMPLE 1

Hyaluronic acid having a molecular weight of 2×10⁶ Da was dissolved in distilled water to give a 1 mass % hyaluronic acid aqueous solution having a pH of 6.0. The pH of the aqueous solution was adjusted to 1.5 with 1N hydrochloric acid. A 2 ml portion of the acidic hyaluronic acid aqueous solution was poured into a 2.5×4.0 cm Petri dish (10 cm²), placed in a refrigerator set at −20° C. for 6 days and thawed at 25° C. to give a sheet of hyaluronic acid gel (hereinafter referred to as “HA gel”). The HA gel was neutralized in 100 ml of phosphate buffered saline, pH 7 for 24 hours and then washed with distilled water sufficiently.

The HA gel was pressed between two plates, swelled with 2 ml of distilled water containing 0.1 mg gentamicin (hereinafter referred to as “GM”) and then freeze-dried to give a 2.5×4.0 cm HA gel sheet containing 0.1 mg GM.

### EXAMPLE 2

The procedure in Example 1 was followed except that 2 ml of distilled water containing 1.0 mg GM was used for swelling before freeze-drying to give a 2.5×4.0 cm HA gel sheet containing 1.0 mg GM.

### EXAMPLE 3

The procedure in Example 1 was followed except that 2 ml of distilled water containing 10.0 mg GM was used for swelling before freeze-drying to give a 2.5×4.0 cm HA gel sheet containing 10.0 mg GM.

### EXAMPLE 4

The procedure in Example 1 was followed except that 2 ml of distilled water containing 100.0 mg GM was used for swelling before freeze-drying to give a 2.5×4.0 cm HA gel sheet containing 100.0 mg of GM.

### COMPARATIVE EXAMPLE 1

The procedure in Example 1 was followed except that 2 ml of distilled water was used for swelling before freeze-drying to give a 2.5×4.0 cm HA gel sheet.

### COMPARATIVE EXAMPLE 2

A fibrin gel containing GM was prepared using “Tisseel” (biological tissue adhesive, imported and sold by Nippon Zoki Pharmaceutical Co., Ltd., manufactured by Immuno (Australia)). To 0.2 ml of the Tisseel fibrinogen solution in the “Tisseel” kit, GM was aseptically added, and then 0.2 ml of the Tisseel thrombin L solution was added for gelation. The fibrin gel was evenly applied onto the surface of a cementless femoral stem having a diameter of 2 mm (manufactured by Zimmer Japan) within about 1 to 3 minutes before the loss of sufficient plastic workability. The cementless femoral stem had a porous coat, like those actually used for total arthroplasty in human, and was freshly prepared before use.

Thus, a cementless femoral stem having a fibrin gel coating containing 10 mg GM was obtained.

### EXAMPLE 5

### Test 1 of GM-Containing HA Gels on the Healing Effect on Osteomyelitis in Mouse Fracture Models

Eight-week-old male Balb/c mice were anesthetized with pentobarbital (20 mg/kg). The knee joints were surgically opened, and the femurs were bared. The bared femurs were fractured transversely with Cooper's scissors, and 105 cells/0.1 ml of MSSA (Staphylococcus aureus, strain S.aureus FDA 209P) were injected into the medullary cavities from the sites of fracture through a 23G needle. A 0.8 mm Kirshner wire (K-wire) wrapped with the HA gel sheet containing 10 mg GM obtained in Example 3 or a bare K-wire with no wrapping was inserted into each knee joint, and the fractures were reduced.

A total of ten mice were divided into two groups of five, and one group was treated with a HA gel sheet containing 10 mg GM obtained in Example 3, while the other was not. They were gassed with CO₂ to death 4 weeks after the operations, and the femurs were extracted and examined for healing of the fractures by SOFTEX radiography (FUJI 100) with X-ray irradiation at 50 KVp and 12 mA for 3 seconds.

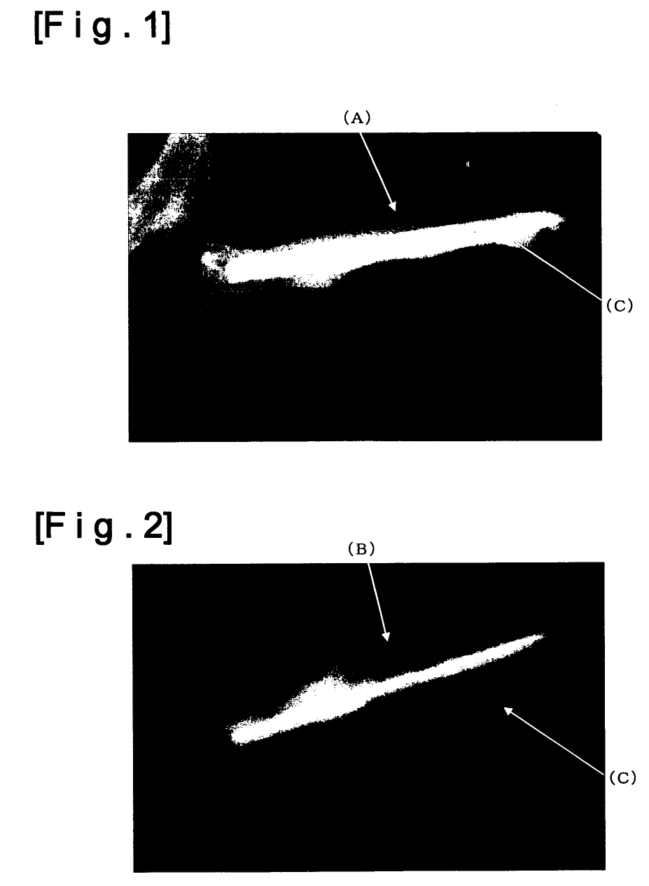

As shown in the radiograph of a mouse femur at 4 weeks in FIG. 1, the fractures had healed at 4 weeks in all of the five mice treated with a HA gel sheet containing 10 mg GM obtained in Example 3, while all of the five mice without treatment with a GM-containing HA gel sheet developed pseudarthrosis with inhibited healing of the fractures as shown in FIG. 2.

Because the HA gel sheet containing 10 mg GM had effective against the lesions of bone infection, it seems to be a useful composition for use in a second operation of total arthroplasty due to infection.

### EXAMPLE 6

Test 2 of GM-Containing HA Gels on the Healing Effect on Osteomyelitis in Mouse Fracture Models

The procedure in Example 5 was followed except that GM-containing HA gel sheets obtained in Examples 1 to 4 were used. A total of twenty mice were divided into four groups of five, and each group was treated with a HA gel sheet containing GM in an amount of 0.1 mg, 1 mg, 10 mg or 100 mg obtained in Example 1, 2, 3 or 4. One week after the operations, they were gassed with CO₂ to death, and the femurs were extracted. Soft tissue samples were collected from around the sites of fractures and weighed on an analytical balance. The samples were homogenized with 1 ml of phosphate buffer (pH 7.0) in Polytoron. The resulting emulsions were diluted with physiological saline by a factor of 1×10³. 100 μl of the diluted emulsion were plated on 5% sheep blood agar (BBL) and incubated at 35° C. for 24 hours, and the colonies were counted. From the weights of the samples in grams, the cell counts per 1 g of sample were calculated.

FIG. 3 indicates that the antibiotic action increased with increasing GM concentration, and at least 10 mg of GM was required for satisfactory antibiotic effect. Because the GM-containing HA gel sheets were effective against the lesions of bone infection, they seems to be useful compositions for use in a second operation of total artroplasty due to infection.

### EXAMPLE 7

Test 3 of GM-Containing HA Gels on the Healing Effect on Osteomyelitis in Mouse Fracture Models

The procedure in Example 6 was followed except that the GM-free HA gel sheet obtained in Comparative Example 1 and the HA sheet containing 10 mg GM obtained in Example 3 were used. The cell counts in soft tissues around the fractures in five mice treated with HA gel sheets containing 10 mg GM obtained in Example 3, were determined at 1, 2, 7 and 14 days respectively in the same manner as in Example 6. The cell counts in soft tissues around the fractures in five mice treated with GM-free HA gel sheets obtained in Comparative Example 1, were determined at 1, 2, 7 and 14 days respectively in the same manner as in Example 6.

As shown in FIG. 4, the cell counts in the group treated with HA gel sheets containing 10 mg GM obtained in Example 3 decreased statistically significantly within 7 days. The cell counts in the group treated with GM-free HA gel sheets obtained in Comparative Example 1 also decreased within 14 days, presumably due to the resistance to the bacteria inherent to the mice. However, the cell counts were significantly lower in the group treated with HA gel sheets containing 10 mg GM obtained in Example 3 than in the group treated with GM-free HA gel sheets obtained in Comparative Example 1.

### EXAMPLE 8

Test 1 of GM-Containing HA Gels on the Healing Effects on Osteomyelitis in Rabbit Osteomyelitis Models

Nine-month-old retired rabbits (Charles River Japan) weighing 3.5 kg on average were anesthetized with pentobarbital (20 mg/kg). The knee joints were surgically opened, and the femurs were bared. Holes with a 2 mm diameter were bored in the knee joints with a drill bar. The rabbits were divided into two groups, and 10⁵ cell/0.1 ml MSSA suspension was injected into the holes in one group, but not in the other group.

Cementless femoral stems (manufactured by Zimmer Japan) having a 2 mm diameter were provided with a porous coating, like those actually used for total arthroplasty in human, and used in the following test.

In the MSSA-injected rabbits, cementless femur stems wrapped with HA gel sheets containing 10 mg GM obtained in Example 3 and cementless femoral stems wrapped with GM-free HA gel sheets obtained in Comparative Example 1 were inserted into the knee joints, and then, the knee joints were reduced. In the MSSA-noninjected rabbits, cementless femoral stems wrapped with HA gel sheets containing 10 mg GM obtained in Example 3 and cementless femoral stems with no wrapping were inserted into the knee joints, and the knee joints were reduced.

A total of 24 rabbits were divided into four groups of six rabbits: an MSSA-injected group implanted with stems wrapped with HA gel sheets containing 10 mg GM obtained in Example 3; an MSSA-injected group implanted with stems wrapped with GM-free HA gel sheets obtained in Comparative Example 3; an MSSA-uninjected group implanted with stems wrapped with HA gel sheets containing 10 mg GM obtained in Example 3; and an MSSA-uninjected group implanted with stems wrapped with no HA gel sheets. All the rabbits were gassed with CO₂ to death 8 weeks after the operations, and the femurs were extracted with the implanted stems and studied radiographically.

Radiographs were taken with SOFTEX (Fuji 100) with X-ray irradiation at 50 KVp and 12 mA for 3 seconds. After the radiographs were taken, the samples were fixed in 70% ethanol at room temperature for 1 day, then in 80% ethanol for 12 hours, in 95% ethanol for 12 hours and in 100% ethanol for 1 day. The samples were embedded in 99% methyl methacrylate monomer (MMA) for 3 days. Finally, the samples were soaked in MMA/Perkadox 16 to obtain hard tissue samples. The rod-like samples were sliced into 0.5 mm thicknesses to make pathologic tissue specimens. The specimens were stained with methylene blue at 60° C. for 8 minutes.

Two of the six MSSA-injected rabbits implanted with GM-free HA gel sheets died. The four survivors showed a significant weight reduction (to an average weight of 2.2 kg at the end of the experiment) as compared with the other groups, and patchy hair loss and bristling, probably because septicemia supervened on osteomyelitis. In contrast, in the other three groups with local administration of GM from the HA gel sheets containing 10 mg obtained in Example 3, all the rabbits were alive at the end of the experiment and did not show a statistically significant weight reduction (average weight: 3.1 kg in the control group, 3.2 kg in the MSSA-uninjected group, and 2.9 kg in the group implanted with HA gel sheets containing 10 mg GM), though they had slightly decreased in weight since the experiment was started, and no change in the fur was observed.

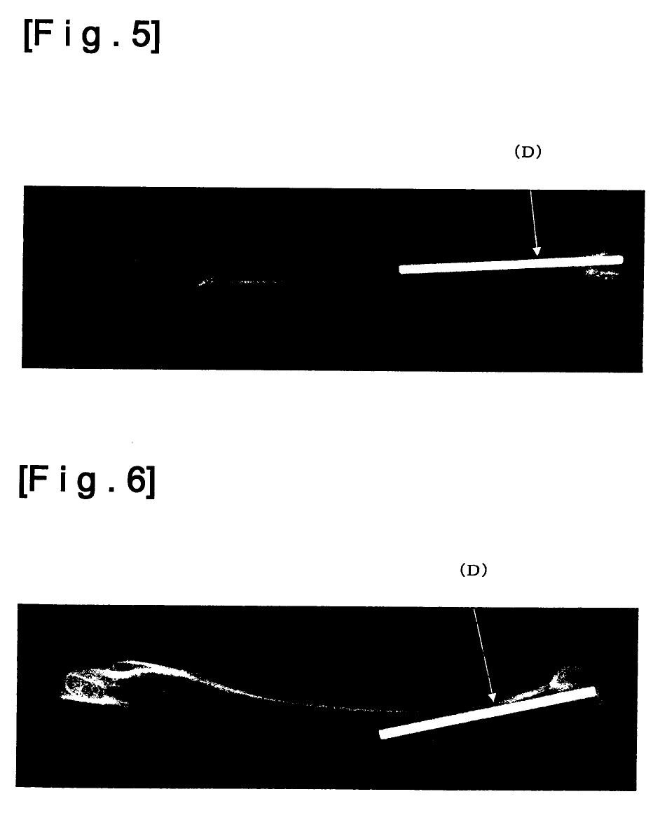

Radiographic studies revealed that no symptoms of osteomyelitis such as bone atrophy or osteolysis were observed in the MSSA-uninjected group implanted with stems, as is evident from the top and lateral radiographs in FIGS. 5 and 6.

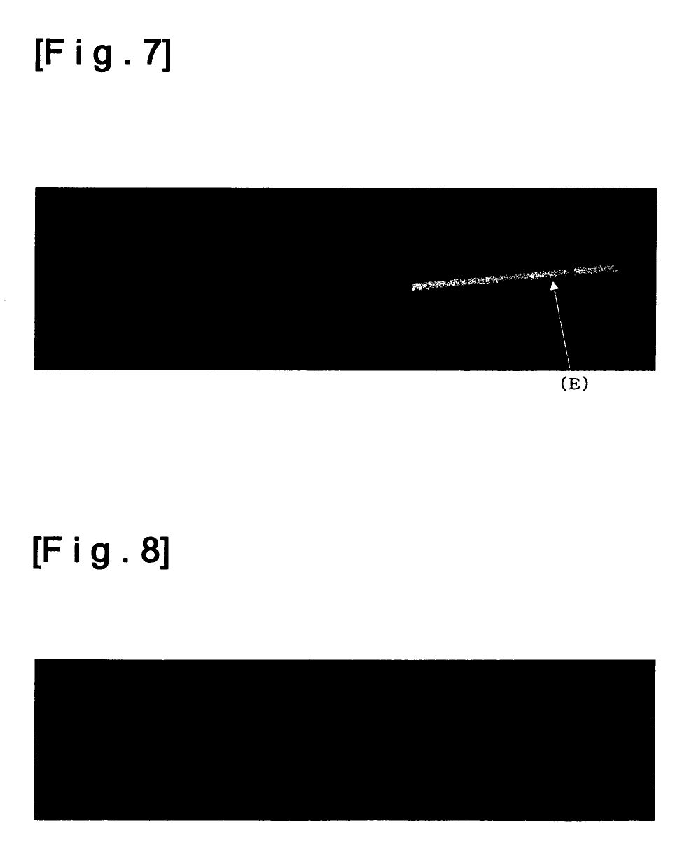

In contrast, in the MSSA-injected group implanted with stems wrapped with GM-free HA gel sheets obtained in Comparative Example 1, obvious bone atrophy and osteolysis were observed around the sites of MSSA injection, presumably because of supervention of osteomyelitis, as is evident from the top and lateral radiographs in FIGS. 7 and 8. In this group, obvious pus exudation from the medullary cavities was observed during femur extraction.

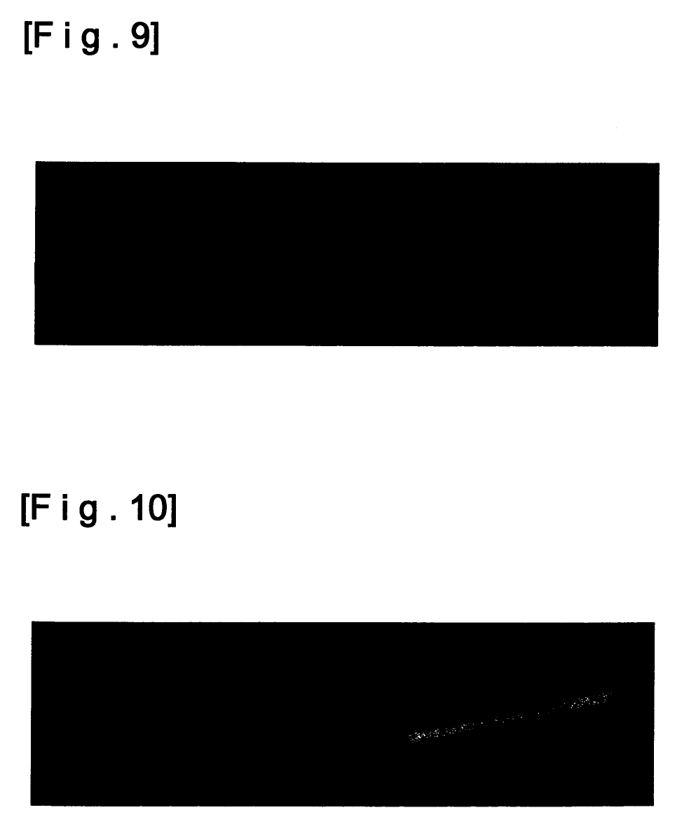

However, as is evident from the top and lateral radiographs in FIGS. 9 and 10, in the MSSA-injected group implanted with stems wrapped with HA gel sheets containing 10 mg GM obtained in Example 3, symptoms of osteomyelitis were obviously suppressed in the radiographs, and no pus exudation was observed at the time of femur extraction.

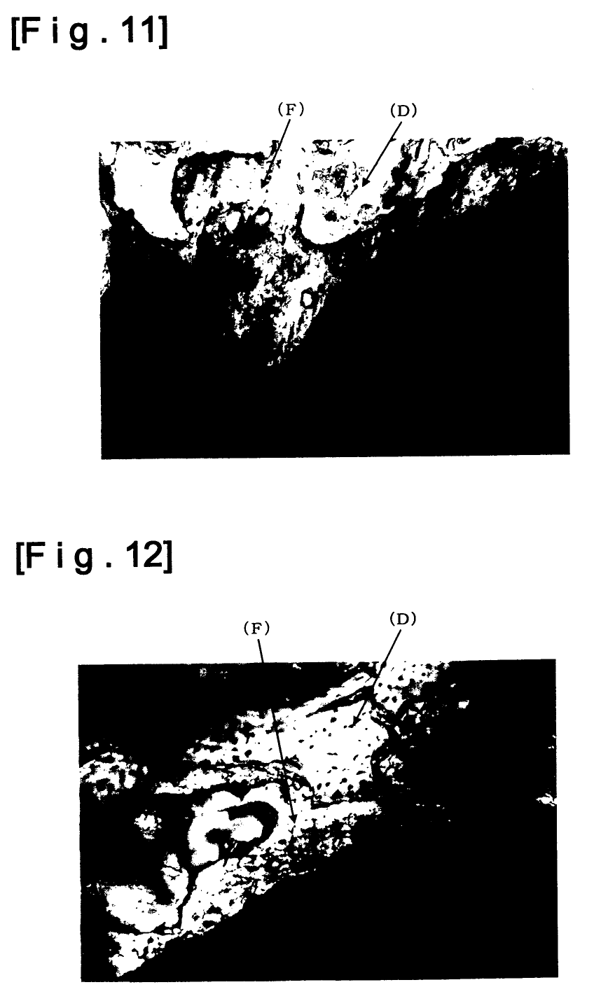

Pathological studies of tissue specimens of the femurs implanted with stems revealed obvious bone extension into the stems in the MSSA-uninjected group implanted with stems, as is evident from FIG. 11.

As shown in FIG. 12, bone extension into stems was also observed in the MSSA-uninjection group implanted with stems wrapped with HA gel sheets containing 10 mg GM obtained in Example 3. This demonstrate that the HA gel sheets do not inhibit bone extension into porous coated stems.

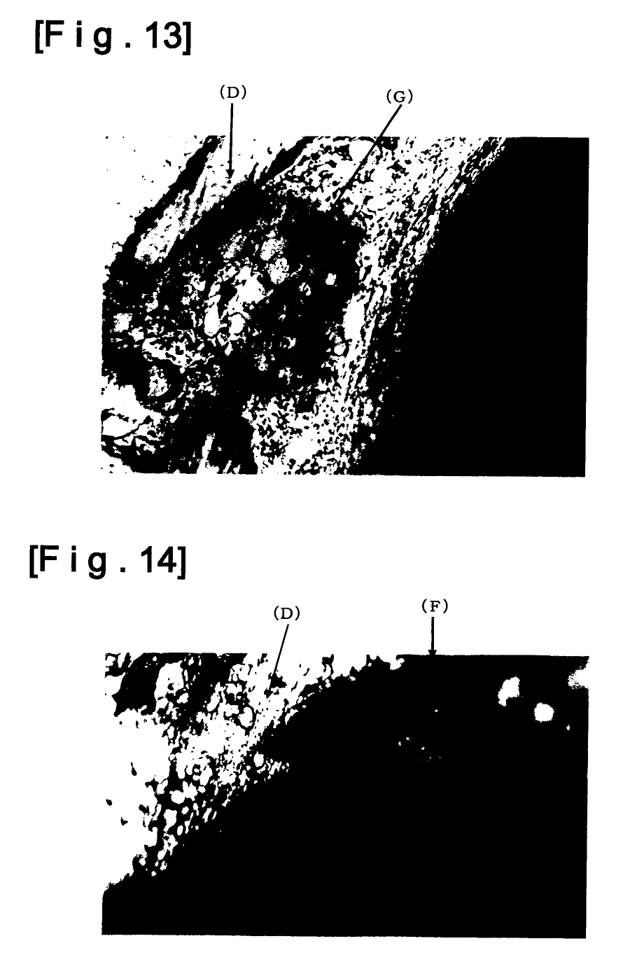

As shown in FIG. 13, the MSSA-injected group implanted with stems wrapped with GM-free HA gel sheets obtained in Comparative Example 1 developed osteolysis around the stems without bone extension, and bacteria cells and abscess formation were observed around the stems.

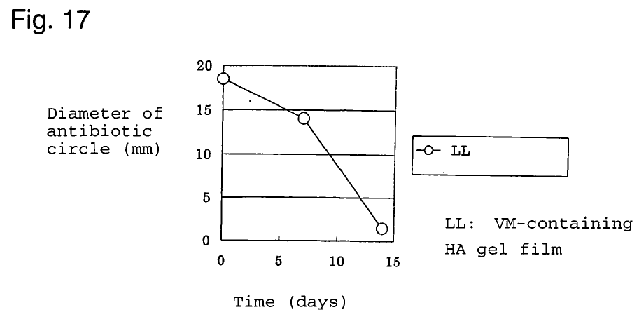

As shown in FIG. 14, in the MSSA-injected group implanted with stems wrapped with HA gel sheets containing 10 mg GM obtained in Example 3, bone extension was observed histologically with no bacteria or abscess formation around the stems.

### EXAMPLE 9

### Test 2 of GM-Containing HA Gels on Healing Effects on Osteomyelitis in Rabbit Osteomyelitis Models

The procedure in Example 8 was followed except that stems coated with a fibrin gel containing 10 mg GM obtained in Comparative Example 2 were used without MSSA injection.

A group of six rabbits were implanted with stems wrapped with HA gel sheets containing 10 mg GM obtained in Example 3 without MSSA injection, and another group of six rabbits were implanted with stems coated with a fibrin gel containing 10 mg GM obtained in Comparative Example 2 without MSSA injection. All the twelve rabbits were gassed with CO₂ to death 8 weeks after the operations, and the femurs were extracted with the implanted stems and studied radiographically.

Pathological studies of the tissue specimens of the femurs implanted with stems wrapped with HA gel sheets containing 10 mg GM obtained in Example 3 revealed obvious bone extension into a stem.

However, in the group implanted with stems coated with a fibrin gel containing 10 mg GM, similar, but less obvious, bone extension into stems was observed. This indicates that HA gel sheets are preferable for bone extension into a porous coated stem to the fibrin gel.

Because the GM-containing HA gel sheet proved effective against infected lesions of bone and did not inhibit bone induction by the cementless artificial joint, it seems to be a useful composition for use in a second operation of total arthroplasty due to infection.

### EXAMPLE 10

Antibiotic Retention Test in Total Arthroplasty Models (HA Gel Sponges)

A HA gel sponge prepared as previously descried was punched with a biopsy trepan to make 4 mm×Ø4 mm samples, and the samples were impregnated with aqueous solutions of 3 mg antibiotics (vancomycin (VM) and minocyclin (MC)) and freeze-dried. For comparison, a GM-containing collagen sponge (manufactured by Biomet) was punched into the same size. Retention of the antibiotics were assayed in the following animal model test.

Japanese white rabbits weighing 3.3 to 3.8 kg were anesthetized by intramuscular injection of ketalar (Sankyo Co., Ltd.) (10 ml/individual), shaved and disinfected with isodine. The vicinity of a knee joint was locally anesthetized with an appropriate amount of xylazine. The skin was cut with a surgical knife, and the articular cartilage was bared. A small hole with a diameter of nearly 2 mm was bored with a hand drill along the femur and filled with a HA gel sponge sample (3 mg antibiotic/4 mm×Ø2 mm sponge), and a metal rod (4 cm×Ø2 mm) was put on the gel sponge sample and sunk into the hole with a hammer. Finally, the articular cartilage and the skin were sutured with nylon thread. This operation was carried out. on both knees at n=4 or more for each antibiotic. After appropriate periods of time, the rabbits were gassed with CO₂ to death, and the femurs were excised and scraped clean. The metal rod was pulled out, and the hole was washed with 1 ml of physiological saline twice, and the washings were combined. The antibiotic activities of the washings were assayed as described below (based on the diameter of antibiotic circle).

S. aureus FDA209 was picked up with a platinum loop from an agar plate culture, preliminarily cultured in 10 ml Heart infusion broth (DIFCO) and spread evenly over a Mueller-Hinton agar in a Petri dish with a spreader. It was incubated with 50 μl of an antibiotic sample to be tested overnight, and the diameter of the antibotic area obtained by the action of the antibiotic was measured.

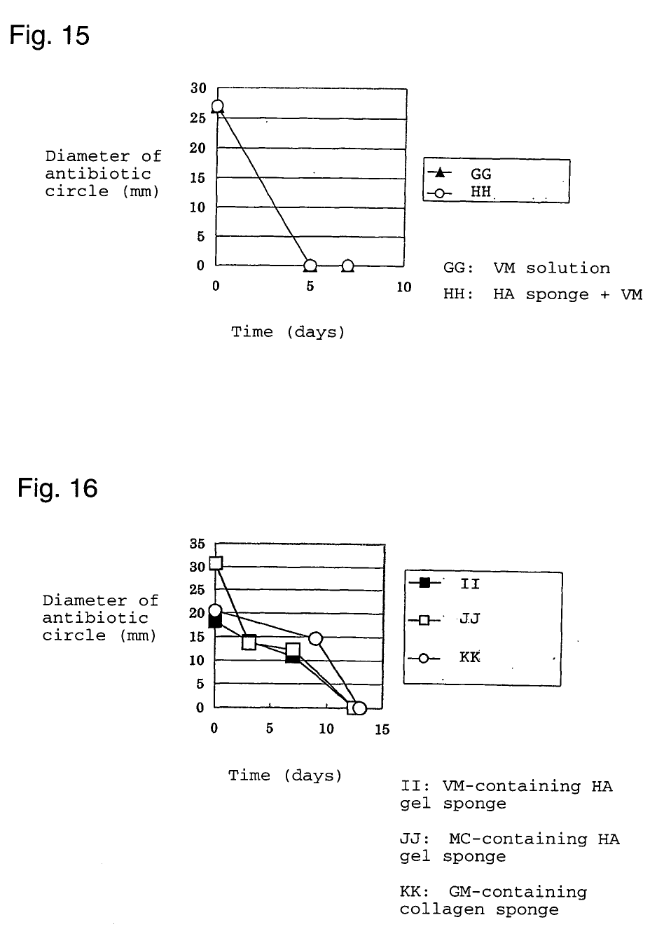

The test results indicate that impregnation into the gel sponge improved retention of the antibiotics in the bone marrow. While when an antibiotic aqueous solution (3 mg/500 μl) without impregnation into a gel and an ungelled HA sponge were used, the antibiotic disappeared from the bone marrow within one week as shown in FIG. 15, all the HA gel sponge samples retained about 50% of the antibiotic activity, as shown in FIG. 16, which is about the same level as the residual antibiotic activity in the GM-containing collagen sponge (manufactured by Biomet).

### EXAMPLE 11

Antibiotic retention test in total arthroplasty models (HA gel films wrapped around metal rods) Tubular HA gel sponges (5 cm×5 mm, inner diameter Ø2 mm) were prepared as previously described and impregnated with an aqueous solution of 3 mg antibiotic (vancomycin) and dried with metal rods (4 cm×Ø2 mm). The resulting HA gel-wrapped metal rods were used for assay of retention of the antibiotic in the following animal model test.

Japanese white rabbits weighing 3.3 to 3.8 kg were anesthetized by intramuscular injection of ketalar (Sankyo Co., Ltd.) (10 ml/individual), shaved and disinfected with isodine. The vicinity of a knee joint was locally anesthetized with an appropriate amount of xylazine. The skin was cut with a surgical knife, and the articular cartilage was bared. A small hole with a diameter of nearly 2 mm was bored with a hand drill along the femur, and a HA gel-wrapped metal rod (4 cm×Ø2 mm) was sunk into the hole with a hammer. Finally, the articular cartilage and the skin were sutured with nylon thread. This operation was carried out on both knees at n=7 or more for each antibiotic. After appropriate periods of time, the rabbits were gassed with CO₂ to death, and the femurs were excised and scraped clean. The metal rod was pulled out, and the hole was washed with 1 ml of physiological saline twice, and the washings were combined. The antibiotic activities of the washings were assayed as described below (based on the diameter of antibiotic circle).

S. aureus FDA209 was picked up with a platinum loop from an agar plate culture, preliminarily cultured in 10 ml Heart infusion broth (DIFCO) and spread evenly over a Mueller-Hinton agar in a Petri dish with a spreader. It was incubated with 50 μl of an antibiotic sample to be tested overnight, and the diameter of the antibotic area obtained by the action of the antibiotic was measured.

The test results indicate that impregnation into the gel sponge improved retention of the antibiotics in the bone marrow. While when an antibiotic aqueous solution (3 mg/500 μl) without impregnation into a gel and an ungelled HA sponge were used, the antibiotic disappeared from the bone marrow within one week as shown in FIG. 15, when a HA gel film was wrapped around a metal rod, about 50% of the antibiotic activity was retained.

### INDUSTRIAL APPLICABILITY

According to the present invention, it is possible to provide a biodegradable composition containing an antibiotic or physiologically active substance for surgical treatment of infection. The biodegradable composition of the present invention containing an antibiotic or physiologically active substance is excellently safe and biocompatible. It shows appropriately sustained release of antibiotics and physiologically active substances and, therefore, has excellent antibiotic and bone regenerating effects. More specifically, the biodegradable polysaccharide and/or polysaccharide gel containing an antibiotic or physiologically active substance is provided as a composition showing an excellent healing effect on bone infections occurring after orthopedic surgery for total arthroplasty in and/or fracture surgery.

1. A composition for treating bone infection, which comprises an antibiotic and a hyaluronic acid and/or a hyaluronic acid gel, wherein the hyaluronic acid gel is crosslinked hyaluronic acid made of hyaluronic acid having a weight average primary molecular weight greater than 800,000.

2. The composition according to claim 1, wherein the bone infection is traumatic bone infection.

3. The composition according to claim 1, wherein the bone infection is hematogenous bone infection.

4. The composition according to claim 1, wherein the bone infection occurs after total arthroplasty and/or fracture surgery.

5-9. (canceled)

10. The composition according to claim 1, wherein the crosslinks in the crosslinked hyaluronic acid are hydrolysable.

11. The composition according to claim 1, wherein the structures of the crosslinks in the crosslinked hyaluronic acid have ester linkages in the structure.

12. The composition according to claim 11, wherein the crosslinks in the crosslinked hyaluronic acid have self-ester linkages in the structure.

13. The composition according to claim 1, wherein the antibiotic is one member selected from the group consisting of gentamicin, vancomycin and minomycin.

14. The composition according to claim 13, which is in the form of one member selected from the group consisting of a sheet, a film, a rod, a sponge, a mass, a fiber, a paste, a gel suspension and a tube.
